(19) United States
(12) Patent Application Publication
(10) Pub. No.: US 2026/0330162 A1
(43) Pub. Date: Oct. 22, 2026

(54) PORTABLE ELECTRONIC DEVICE AND METHOD OF READING A MACHINE-READABLE IDENTIFIER

(71) Applicant: SANOFI, Paris (FR)

(72) Inventors: Stefan ALT, Frankfurt am Main (DE); Michael HELMER, Frankfurt am Main (DE)

(21) Appl. No.: 19/481,306

(22) PCT Filed: May 2, 2024
(86) PCT No.: PCT/EP2024/062011

§ 371 Date: Nov. 4, 2025

(30) Foreign Application Priority Data
May 5, 2023 (EP) 23315164.6

(51) Int. Cl. G06K 7/10

(52) U.S. Cl. CPC G06K 7/10297; G06K 7/10366

(57) ABSTRACT

A portable electronic device is disclosed comprising a camera configured to capture an image of a medical device bearing a machine-readable identifier, an electronic reader configured to read the machine-readable identifier when aligned therewith, an electronic display configured to present the captured image, and a processor operatively coupled to the camera and the display. The processor is configured to generate and display, on the electronic display, a virtual outline corresponding to the medical device, wherein the virtual outline is arranged to align with and/or overlap the captured image when the electronic reader is aligned with the machine-readable identifier. In certain embodiments, the device further comprises or accesses an electronic device identifier containing spatial image offset information indicative of a spatial offset between the camera and the electronic reader. The spatial offset information is usable to calibrate the alignment or overlap between the captured image and the virtual outline.

### CROSS-REFERENCE TO RELATED APPLICATIONS

The present application is the national stage entry of International Patent Application No. PCT/EP2024/062011, filed on May 2, 2024, and claims priority to European Application No. EP 23315164.6, filed on May 5, 2023, the disclosures of which are incorporated herein by reference.

### TECHNICAL FIELD

The present disclosure relates to the field of medical devices and specifically to user assistance for using or operating medical devices, such as injection devices, and, more specifically, to a portable electronic device configured to assist a user in using a medical device. The disclosure also relates to a system comprising a medical device and a portable electronic device and to a method of reading a machine-readable identifier provided in or on a medical device.

### BACKGROUND

Drug delivery devices allowing for multiple dosing of a required dosage of a liquid medicinal product and further providing administration of such liquid drug to a patient, are as such well known in the prior art. Generally, such devices have substantially the same purpose as that of an ordinary syringe. Typically, a medicinal product to be administered is provided in a cartridge having a moveable piston or bung mechanically interacting with a piston rod of a drive mechanism of the drug delivery device. By applying thrust to the piston, a certain and pre-defined amount of the medicinal fluid is expelled from the cartridge.

In the field of home medication or self-medication, a user or patient may be provided with a medical device, such as an injection device, e.g. for executing an injection procedure. Such medical devices may be implemented mechanically, or electro-mechanically.

Some medical devices, such as injection pens, may be mechanically implemented and may be manually operated by a user for injecting of a dose of a medicament. Some injection devices are implemented as disposable injection devices. They may be equipped with a prefilled medicament container. The medicament container may be preassembled inside the injection device. Such injection devices may be handed out to patients or users in a condition or state, in which the device is ready to use. Here, the device may represent a drug-device combination and might be intended or configured to become discarded after use.

Medical devices configured and intended for home medication or self-medication through a user or patient may be electronically coupled with external or portable electronic devices, such as smart phones, tablet computers, smart watches, or so-called auxiliary or add-an devices configured to cooperate with the medical device or injection device. Such external devices may assist a user in conducting administering of a medicament, e.g. in form of dose injection procedure as well as to monitor operation of the injection device and/or of a medicament administering injection procedure. Portable electronic devices, such as smart phones, tablet computers or smartwatches may be operable to electronically pair with the medical device, e.g. with the injection device. Portable electronic devices or add-on devices, particularly configured for detachably fastening to a medical device or injection device may provide an automated or semi-automated logging or tracking of numerous dose administering or dose injection procedures.

An electronic coupling between a portable electronic device and a medical device or injection device may be beneficial to record and/or to store a single or repeated uses of the medical device or injection device.

Medical devices or medicament containers, such as injection devices, may be provided with a unique identifier, e.g. being indicative of the type, the amount, the LOT number of a medicament charge, the production date, the production site and/or a best before date of a medicament located or stored in the respective medical device. Some portable electronic devices may offer an automated or semi-automated readout of the identifier of the medical device so as to obtain the respective information about the medicament provided with or stored in the medical device or medicament container.

Capturing or obtaining of information provided by an identifier of a medical device, such as an injection device may be provided electronically. Hence, the identifier of a medical device or of a medicament container associated with the medical device may be operable or suitable for machine reading and may be readout or captured autonomously by the portable electronic device. An automated or machine-based readout of a machine-readable identifier of a medical device or medicament container may require a rather specific relative arrangement or orientation between the portable electronic device and the medical device.

Depending on the technologic implementation of machine-reading, it may be required that the portable electronic device is held in a well-defined position, distance and/or orientation relative to the medical device in order to enable an automated or machine-based readout of information stored in the machine-readable identifier of the medical device.

Especially in situations, where the machine-readable identifier is not directly apparent on an outside surface of the medical device or in situations, where a patient or user may have difficulties in correctly orienting or positioning the portable electronic device relative to the medical device it may be quite difficult for a user or patient to bring or to arrange the portable electronic device in a predefined position or orientation relative to the medical device in order to enable the automated or machine-based readout.

It is therefore desirable to provide improvements to a portable electronic device and to a method of reading a machine-readable identifier provided in or on a medical device. It would be of particular benefit to simplify the process of correctly aligning, positioning or orienting a portable electronic device relative to a medical device in order to enable a fast, reliable and complete reading of a machine-readable identifier as provided in or on a medical device. It would be of particular benefit to assist a user in correctly orienting or positioning the portable electronic device relative to the medical device to enable the automated or machine-based readout of the machine-readable identifier.

### SUMMARY

In one aspect, the present disclosure relates to a portable electronic device. The portable electronic device comprises a camera, which is operable to capture an image of a medical device. The medical device is provided with a machine-readable identifier. The portable electronic device further comprises an electronic reader, which is operable to read the machine-readable identifier when aligned with the machine-readable identifier. In the present context, the electronic reader is aligned with the machine-readable identifier when the electronic reader is at least in one of a predefined distance relative to the machine-readable identifier, a predefined relative orientation relative to the machine-readable identifier and a predefined position relative to the machine-readable identity. In other words, the electronic reader is operable to read the machine-readable identifier when the electronic reader is within a reading distance and is correctly oriented, e.g. in a reading orientation relative to the machine-readable identifier. Otherwise, and when the electronic reader should be outside a reading distance or reading orientation, the electronic reader may be inoperable to read the machine-readable identifier of the medical device.

The portable electronic device further comprises an electronic display. The electronic display is operable to visualize the captured image of the medical device. The portable electronic device also comprises a processor, which is connected to the camera and which is further connected to the electronic display. The processor may be operable to process image data as generated and obtained from the camera and to use the image data to visualize the images captured by the camera on or in the electronic display.

The processor is further operable to display a virtual outline of the medical device on the electronic display, such that the virtual outline aligns and/or overlaps with the captured image of the medical device on the electronic display when the electronic reader is aligned with the machine-readable identifier. In other words, the processor is operable to display the virtual outline of the medical device on the electronic display, such that, when the electronic reader is aligned with the machine-readable identifier, the virtual outline aligns and/or overlaps with the captured image on the electronic display.

In this way the portable electronic device is provided with an assistance function by way of which the camera, the processor and the electronic display of the portable electronic are used to bring the portable electronic device into alignment, i.e. into a reading distance and/or a reading orientation relative to the machine-readable identifier, such that when the portable electronic device is correctly aligned with the machine-readable identifier of the medical device the electronic reader of the portable electronic device is enabled to read or to capture the machine-readable identifier as provided in or on the medical device.

By illustrating the virtual outline of the medical device on the electronic display of the portable electronic device concurrently with a captured image of the medical device, provides a rather intuitive approach to correctly align and/or to correctly arrange the portable electronic device relative to the medical device in order to enable an automated or machine-based readout of data from the machine-readable identifier. Here, the captured image is captured by the camera of the portable electronic device and may represent a live-view of the momentary configuration or position of the medical device.

In other words, the portable electronic device with its concurrent visual illustration of the virtual outline of the medical device together with the captured image of the medical device, which is momentarily captured by the camera of the portable electronic device, provide a rather intuitive approach to assist a user in correctly aligning or orienting the portable electronic device and the medical device relative to each other.

In some examples, the portable electronic device provides a kind of an augmented reality to a user. The virtual outline as provided on the display of the portable electronic device may represent a target position and/or target orientation for the medical device, which when reached by the medical device ensures, that the electronic reader is correctly aligned with the machine-readable identifier of the medical device.

In some examples, the virtual outline as displayed on the electronic display of the portable electronic device is rather static. It may be artificially generated on the electronic display. In other examples the virtual outline is a dynamic virtual outline, which due to varying situations or varying mutual arrangements of the medical device relative to the portable electronic device may be also subject to visual modifications.

In some examples, the camera and the electronic reader of the portable electronic device are located at different positions of the portable electronic device. They may be provided and arranged relative to each other at a predefined spatial offset. Insofar, arranging and aligning the camera of the portable electronic device to the machine-readable identifier may be insufficient to bring the electronic reader of the portable electronic device in the predefined or required alignment with the machine-readable identifier.

According to another example, the portable electronic device comprises an electronic device identifier containing or providing a spatial image offset information being indicative of a spatial offset between the camera and the electronic reader. The electronic device identifier may be stored in a memory or storage of the portable electronic device. The electronic device identifier may be indicative of a spatial image offset information and hence of a spatial offset between the camera and the electronic reader of the medical device. The spatial image offset information may be also indicative about a relative position and/or relative orientation between the camera and the electronic reader, e.g. also in relation to the outline or housing geometry of the portable electronic device.

Specifically, the spatial image offset information represents a hardware specific configuration of the portable electronic device and may differ between different portable electronic devices. Hence, with some manufacturers of portable electronic devices or with some models of portable electronic devices the camera and the electronic reader may almost axially overlap. With other manufacturers or models of portable electronic devices there may be a rather large spatial offset between the camera and the electronic reader of the portable electronic device. The electronic device identifier contains at least information about the manufacturer and the specific type of the portable electronic device. For each unique electronic device identifier there may be provided respective spatial image offset information being indicative of a spatial offset and/or of a relative position or orientation between the camera and the electronic reader of the respective portable electronic device.

The processor of the portable electronic device may be operable to read and/or to process the spatial image offset information in order to calibrate the visual guiding function as provided by the processor. Hence, for a first type of a portable electronic device with a first electronic device identifier there is provided a first spatial image offset information, which when processed by the processor leads to the generation of a first virtual outline of the medical device in a first position and/or first orientation on the electronic display of the portable electronic device.

With another portable electronic device, e.g. a second portable electronic device of a different type, there may be provided a second electronic device identifier containing or representing second spatial image offset information, which is likewise processable by the processor. The respective second spatial image offset information may be processed by the processor of the respective portable electronic device to generate a second virtual outline of the medical device, which may differ in at least one of a size, position or orientation on the electronic display compared to at least one of the size, the position or orientation of the first virtual outline of the medical device.

The electronic device identifier enables to implement a respective computer program, a software or software application, e.g. an app for the portable electronic device, which is operable with a large variety of different types of portable electronic devices, e.g. to operate with portable electronic devices that distinguish by their hardware configuration, in particular by the position, orientation or distance of a camera relative to the electronic reader.

By way of the electronic device identifier the respective computer program or software executed by the portable electronic device can be universally adapted to different types of portable electronic devices.

In other words, and by way of the electronic device identifier, the processor of the respective portable electronic device can be individually calibrated to the respective and rather specific hardware implementation of the portable electronic device. In this way, the augmented reality guidance function for assisting a user in correctly positioning or aligning the portable electronic device relative to a medical device can be deployed with a large variety of portable electronic devices of different manufacturers or of different type.

According to another example, the portable electronic device comprises a memory. The memory is operable or configured to store spatial device offset information being indicative of a spatial offset between the machine-readable identifier and at least one of a visual mark on the medical device and an outline of the medical device. In some examples the visual mark and/or the outline of the medical device may be recognizable in the captured image visualized on the electronic display of the portable electronic device. With the further information about a spatial offset between the machine-readable identifier and at least one of the visual mark and the outline of the medical device, the visual mark and/or the outline of the medical device can be used to define or to locate the machine-readable identifier on or inside the medical device. In other words, a well-defined or predefined spatial device offset information indicating a spatial offset between the machine-readable identifier and at least one of the visual mark and the outline or geometry of the medical device itself can be used to locate the machine-readable identifier on the basis of recognizing at least one of the visual mark, the outline and/or the outer geometry of the medical device in the captured image on the electronic display.

According to another example, the processor of the portable electronic device is operable to display the virtual outline of the medical device on the electronic display with respect to at least one of the spatial image offset information and the spatial device offset information. Here, at least one of the spatial image offset information and the spatial device offset information, which is characteristic for the type of the portable electronic device and/or of the type of the medical device, respectively, can be used to provide a precise calibration for a spatial alignment functionality as provided by the portable electronic device. In particular, and for different portable electronic devices the respective spatial image offset information can be used to correctly display or illustrate the virtual outline of the medical device on the electronic display such that when aligned or overlapping with the captured image on the electronic display, the electronic reader of the respective portable electronic device is correctly aligned with the machine-readable identifier of the medical device.

In the same or like manner the spatial device offset information can be used to provide a correct alignment between the electronic reader and the machine-readable identifier for different types of medical devices. Here, the portable electronic device can be least provided with generic information about the spatial device offset information. The portable electronic device is intended for use with a type or class of medical devices that match with the spatial device offset information stored in the memory of the portable electronic device.

In some examples, the portable electronic device may be configured or preconfigured for use with only one or several types of medical devices, each of which being assigned with identical spatial device offset information between the machine-readable identifier and at least one of a visual mark on the medical device and an outline of the medical device. Provided that the portable electronic device is always used with this type or class of medical devices the spatial device offset information stored in the memory of the portable electronic device can be used in addition or instead of the spatial image offset information in order to provide or to establish a correct alignment between the machine-readable identifier and the electronic reader on the basis of a spatial overlap or alignment between the virtual outline and the captured image of the medical device.

According to another example, the processor is operable to recognize at least one of a position and an orientation of at least one of the medical device and the electronic identifier in the captured image as a so-called recognized item. Insofar, the processor may be operable to autonomously or automatically recognize at least one of the medical device, e.g. an outline or geometry of the medical device, and the electronic identifier in the captured image. By way of such an automated or semi-automated item recognition the user can be further assisted in arranging or aligning the portable electronic device relative to the medical device; or vice versa.

The automatic and hence processor-assisted recognition of at least one of the electronic identifier and the orientation or position of the medical device can be further used by the processor and/or by the user to facilitate the process of correctly aligning the portable electronic device relative to the medical device.

In some examples, the processor may be also operable to recognize a visual mark as provided on the medical device, which recognition may be then further used by the processor and/or by a user of the portable electronic device to correctly align the portable electronic device relative to the medical device, such that the electronic reader is aligned with the machine-readable identifier in a pre-defined manner, in which the electronic reader is operable to read information from the machine-readable identifier.

According to a further example, the processor is operable to modify an appearance of the virtual outline on the electronic display depending on a degree of alignment and/or depending on a degree of overlapping between the recognized item and the virtual outline. In this way, a further user assistance can be provided by the processor to facilitate the user-conducted mutual alignment of the portable electronic device and the medical device.

The appearance of the virtual outline can be modified in a number of different ways. The modification of the visual appearance of the virtual outline further helps to guide or to assist a user to correctly align the portable electronic device relative to the medical device. It provides an immediate visual feedback to the user in response to a user-induced movement of the medical device relative to the electronic device.

The modification of appearance of the virtual outline may be indicative of a distance mismatch and/or orientation mismatch between the machine-readable identifier of the medical device and the electronic reader of the portable electronic device. Varying of a distance and/or varying of an orientation of the machine-readable identifier relative to the electronic reader, which can be detected through evaluation of the recognized item by the processor, may induce a modification of appearance of the virtual outline of the medical device on the electronic display. The modification of appearance of the virtual outline may be directly indicative to a user, that the degree of alignment and/or a degree of mutual overlap between the electronic reader and the machine-readable identifier has been significantly changed, e.g. reduced or increased.

According to another example, the processor is operable to modify at least one of a structure, a color, a brightness, a contrast and a temporal appearance of the virtual outline in response to a variation of the degree of alignment and/or in response to a variation of an overlapping between the recognized item and the virtual outline. For instance, and with a low degree of alignment or overlapping between the recognized item and the virtual outline, the virtual outline may appear with a comparatively large degree of brightness and/or contrast on the display. With an increasing degree of alignment or overlapping, the brightness and/or the contrast of the virtual outline may decrease.

In another example, also the color of the virtual outline may be subject to modification. E.g. with a comparatively low degree of alignment or overlapping with the recognized item, the virtual outline may be represented in a per-defined color, e.g. in red. As soon as the degree of alignment and/or overlapping between the recognized item and the virtual outline increases the color thereof may change towards yellow. With a further and almost perfect or intended alignment between the recognized item and the virtual outline the color of the virtual outline may change to green, thereby intuitively indicating to the user, that a predefined alignment or orientation of the machine-readable identifier relative to the electronic reader has been reached.

In the same way, also the brightness and/or contrast as well as a geometric structure of the virtual outline may be subject to a stepwise or continuous change as the degree of alignment and/or the degree of spatial overlapping between the recognized item in the captured image and the virtual outline as provided on the electronic display changes.

According to another example, the processor of the portable electronic device may be further operable to display a transparent or semi-transparent virtual image of the medical device on the electronic display, such that, when the electronic reader is aligned with the machine-readable identifier the virtual image aligns and/or overlaps with the captured and reproduced image on the electronic display.

By way of a transparent or semi-transparent virtual image there can be even provided a three-dimensional guiding functionality for the user to arrange the portable electronic device in a well-defined and/or predefined orientation or position relative to the medical device with respect to all three spatial dimensions.

With a transparent or semi-transparent virtual image, it is also possible to modify a three-dimensional position or orientation of the medical device relative to the portable electronic device. With the medical device and hence, with a three-dimensional object, e.g. comprising a pen-type injector, the housing of the device may be of elongated tubular shape. Here, the electronic reader may be provided at a well-defined tangential or circumferential position on the circumference of the tubular shaped housing of the medical device. A correct reading of the machine-readable identifier may require a specific orientation and hence a specific rotational position of the medical device relative to its longitudinal axis relative to the portable electronic device.

With the transparent or semi-transparent virtual image and with the requirement that the portable electronic device is to be used with a particular type or class of medical devices, hence when having respective spatial device offset information stored in the memory of the portable electronic device, the transparent or semi-transparent virtual image can be presented on the electronic display, e.g. concurrently with the visualization of the momentarily captured image as obtained by the camera of the portable electronic device.

The transparent or semi-transparent virtual image may also comprise or reproduce a visual mark as provided on the outside of the medical device. Here, and e.g. on the basis of such a visual mark the user or patient may conduct a required rotation of the medical device relative to the portable electronic device, such that not only the virtual outline of the medical device overlaps with the recognized item and hence with the captured image of the medical device but that also the medical device is rotated or oriented in a predefined way such that the machine-readable identifier typically faces directly towards the electronic reader of the portable electronic device.

In some examples, the visual mark may be included in them transparent or semi-transparent virtual image and may characterize at least one of the medical device and the medicament container of the medical device. By way of the visual mark, the medical device may distinguish from other medical devices. In the same or like manner, and by way of their visual mark, also the medicament container may distinguish from other medicament containers. Even when accommodated or located inside the housing of the medical device the medicament container may be visible from outside, at least to such an extent, that the visual mark as provided on the medicament container is visible and can be hence captured by the camera of the portable electronic device.

It may be provided that the visual mark on the outside of the medical device should be brought in an overlapping configuration with the respective transparent or semi-transparent virtual visual mark of the transparent or semi-transparent virtual image of the medical device.

According to another example, the electronic reader of the portable device comprises a near field communication (NFC) transceiver. A near field communication transceiver requires a well-defined position and/or or orientation of the medical device with its machine-readable identifier relative to the electronic reader. Typically, the machine-readable identifier should be directly facing the electronic reader; and vice versa. In some examples the spatial distance between the electronic reader and the machine-readable identifier should be less than 5 cm, less than 4 cm, less than 3 cm, less than 2 cm or even less than 1 cm or less than 5 mm.

Also, a transversal spatial offset between the electronic reader and the machine-readable identifier should be less than 5 cm, 4 cm, 3 cm, 2 cm or less than 1 cm.

In this way, and when the electronic reader, e.g. in form of a NFC transceiver, is within a predefined distance or range, a precise and reliable reading of electronically stored information of the machine-readable identifier can be read by the electronic reader. With the electronic reader comprising a NFC transceiver the machine-readable identifier is typically implemented as a NFC tag, which is provided on or inside the housing of the medical device. It may be provided on or inside a medicament container, such as a cartridge filled with a pharmaceutically active substance.

In another example, the electronic reader comprises a RFID reader and the machine-readable identifier comprises a respective RFID tag.

The machine-readable identifier may be implemented as a passive machine-readable electronic circuit, which is void of an own supply of electric energy.

In another example, the machine-readable identifier may comprise an optical code or an optically readable identifier. Here, the machine-readable identifier may comprise a two-dimensional visual code, e.g. on an outside surface of a housing of the medical device.

In another aspect, the present disclosure relates to a system comprising a medical device and a portable electronic device as described above. The medical device comprises a housing and machine-readable identifier on or inside the housing. The machine-readable identifier may be provided on an outside surface of the housing. In some examples the machine-readable identifier may be located inside the housing and may be concealed by the housing. It may not be visible from outside the housing. The portable electronic device comprises the electronic reader as described above, which is operable to read the machine-readable identifier of the medical device when aligned with the machine-readable identifier.

Alignment with the machine-readable identifier means that the distance between the machine-readable identifier and the electronic reader is less than or equal a predefined transmission range of the electronic reader. In some examples it may be also required that the electronic reader is aligned with a pre-defined orientation of the machine-readable identifier. With the electronic reader implemented as a NFC transceiver, the alignment mismatch between the machine-readable identifier and the electronic reader may be less than 5 cm, less than 4 cm, less than 3 cm, less than 2 cm, less than 1 cm or even less than 5 mm.

The system comprises the portable electronic device as described above. Insofar, all features, effects and benefits as described above in regards to the portable electronic device equally apply to the system comprising the medical device and the portable electronic device.

In a further example of the system, the medical device comprises an injection device. The injection device may be implemented as a hand-held injection device. It may comprise a medicament container filled with a liquid medicament and may further comprise an injection needle in fluid connection with an interior of the medicament container. In some examples the injection device comprises at least one of a plunger and a piston slidably arranged inside the medicament container. The medicament container may be implemented as a cartridge filled with the liquid medicament and comprising a tubular-shaped barrel, inside which the plunger or piston is longitudinally movable. By displacing the plunger or piston relative to the barrel a well-defined amount of the medicament, e.g. a dose of the medicament, can be expelled through an outlet of the medicament container, which is typically located at or near a distal end of the medicament container.

In some examples, the injection device comprises an injection pen. The injection pen may be implemented as an all-mechanically operated device, where a user has to exert a dispensing force sufficient to drive or urge the plunger or piston in distal direction to expel the dose of the medicament from the medicament container.

In some examples, the medical device or injection device is implemented as a so-called auto injector. Here, a user may only have to bring the housing of the injection device in mechanical contact with the skin and to initiate a dispensing procedure, e.g. by activating a trigger and/or by pressing or moving the housing of the injection device against or towards the injection site on the skin of the patient. With an autoinjector, a needle configured to puncture or to pierce the tissue of the patient may automatically advance and may enter the skin of the patient. With an autoinjector and after having pierced the skin of the patient, an automatic injection procedure may start or continue by way of which a well-defined amount of medicament, i.e. a dose, is injected through the injection needle into the biological tissue, e.g. into the tissue of the patient.

In some examples, the injection device comprises an injection pen allowing or providing setting and dispensing of doses of variable size. In some examples the injection device is configured for setting and injecting multiple doses of equal or of different size, wherein the user himself may adjust the size of the dose to be injected. In other examples the injection device is fixed dose device. It may be implemented as a disposable device with the medicament and/or the medicament container readily stored and/or assembled inside the housing of the injection device.

In further examples, the injection device is a reusable injection device offering a replacement of an empty product cartridge by a cartridge or medicament container filled with a medicament.

In some examples, the medical device itself, e.g. the housing of the medical device, which is configured to receive or to accommodate a medicament container or cartridge, is provided with the machine-readable identifier. In other examples it is the medicament container to be arranged inside the medical device, which is provided with the machine-readable identifier. In this way, the information stored or provided by the machine-readable identifier is indicative of characteristics of the medicament stored therein.

According to another example, the medical device comprises a medicament container, which is filled with a medicament. The injection device or medical device may be implemented as a disposable medical device, which is intended for a single or for multiple uses and which after consumption or use of the medicament initially stored therein is intended to become discarded in its entirety.

According to a further aspect, the present disclosure relates to a method of reading a machine-readable identifier provided in or on a medical device with a portable electronic device. Hence, the portable electronic device is used to read the machine-readable identifier as provided in or on the medical device, wherein the medical device is typically implemented as an injection device. The portable electronic device comprises a camera, an electronic reader and an electronic display. The method further comprises the steps of capturing an image of the medical device with the camera of the portable electronic device, displaying a virtual outline of the medical device on the electronic display, visualizing the captured image of the medical device on the electronic display and moving the medical device relative to the portable electronic device to align and/or to at least partially overlap the captured image with the virtual outline of the medical device on the electronic display, thereby bringing the electronic reader into one of a reading distance or reading orientation with respect to the machine-readable identifier.

In other words, the virtual outline of the medical device is brought in alignment and/or in a partial overlap with the captured image to bring the electronic reader into one of a reading distance or reading orientation with respect to the machine-readable identifier. Typically, the movement, orientation or alignment of the portable electronic device relative to the medical device is assisted or guided by displaying of the virtual outline of the medical device on the electronic display concurrently with the momentarily captured image of the medical device. Insofar, the virtual outline displayed on the electronic display provides a kind of an augmented reality for bringing the electronic reader of the portable electronic device in reading distance or reading orientation with respect to the machine-readable identifier as provided in or on the medical device.

The method of reading the machine-readable identifier is typically conducted with a portable electronic device after or with a system as described above. Insofar, all features, effects and benefits as described above in connection with the portable electronic device and the system equally apply to the method of reading the machine-readable identifier; and vice versa.

According to another example, the method further comprises the step of obtaining or acquiring of at least one of a spatial image offset information and a spatial device offset information. Here, the spatial image offset information is indicative of a spatial offset between the camera and the electronic reader of the electronic device. The spatial device offset information is in turn indicative of a spatial offset between the machine-readable identifier and at least one of a visual mark on the medical device and an outline of the medical device.

The method further comprises the step of displaying the virtual outline of the medical device on the electronic display with respect to at least one of the spatial image offset information and the spatial device offset information. The spatial image offset information is typically characteristic of a particular type or hardware configuration of the portable electronic device actually used with the method of reading the machine-readable identifier.

Generally, a large variety of differently configured portable electronic devices can be used to read the machine-readable identifier as provided in or on the medical device. With the spatial image offset information, which may be characteristic for each available portable electronic device, the position and/or orientation of the machine-readable identifier on the electronic display can be adapted and modified in accordance to the device specific configuration of the portable electronic device. In this way, the method can be universally used with a large variety of different portable electronic devices, which distinguish by their hardware configuration, and which in particular distinguish by the geometric or spatial offset between the camera and the electronic reader.

In the same way or concurrently, also spatial device offset information of a particular medical device can used by the method of reading the machine-readable identifier. There, the spatial device offset information may be indicative of a spatial offset between the machine-readable identifier and at least one of a visual mark on the medical device and an outline of the medical device.

The spatial device offset information and hence a spatial offset between the machine readable identifier and one of the visual mark of the medical device and the outline of the medical device may be of particular use to provide a correct alignment between the electronic reader and the machine-readable identifier. The spatial device offset information may be indicative that the machine-readable identifier is provided in a specific position relative to or in relation to the outline of the medical device and a visual mark on the medical device.

For a given type or class of medical devices, the spatial device offset information may be always the same and the portable electronic device may be particularly configured to communicate with or to read-out a machine-readable identifier of this particular type or class of medical devices. With the spatial device offset information available to the portable electronic device, e.g. per default or stored in a memory thereof, there can be provided a precise alignment of the machine-readable identifier relative to the electronic reader on the basis of the spatial device offset information.

In another example, the method also comprises the step of recognizing at least one of the position and an orientation of at least one of the medical device and the electronic identifier in the captured image as a recognized item. Here, the processor may be provided with image processing capabilities and may automatically recognize or track at least one of a predefined spatial pattern of the electronic identifier and/or an outline of the medical device and may assign the recognition of the electronic identifier and/or of the position or orientation of the medical device in the captured image as a recognized item. In this way, the processor may be operable to identify or to recognize the machine-readable identifier and hence the class or type of the medical device as such in the captured image and may thus provide an enhanced functionality to bring the captured image of the medical device in suitable overlapping or alignment with the virtual outline as provided concurrently on the electronic display.

According to another example, the method also comprises the step of modifying an appearance of the virtual outline of the electronic display depending on a degree of alignment and/or depending on a degree of overlapping between the recognized item and the virtual outline. The modification of the appearance of the virtual outline can be governed by the degree of alignment and/or by the degree of overlapping between the recognized item and the virtual outline.

According to a further aspect, the present disclosure relates to a computer program comprising computer executable instructions, which when executed by a processor of of the portable electronic device causes the processor to capture an image of the medical device with the camera of the portable electronic device. The computer executable instructions further cause the processor to display a virtual outline of the medical device on the electronic display and to visualize the captured image of the medical device on the electronic display concurrent with the virtual outline of the medical device.

In some examples, the computer program is executable by the processor of an electronic device as described above and is configured to execute or to conduct the individual steps of a method of reading the machine-readable identifier of the medical device as described above. Insofar, all features, effects and benefits as described above in connection with the portable electronic device, the system comprising the medical device and the portable electronic device and all features, effects and benefits as described above in connection with the method of reading the machine-readable identifier as provided in or on a medical device, equally apply to the computer program with its computer executable instructions; and vice versa.

The computer program may be deployed or installed as an app on a portable electronic device, such as a smartphone, a tablet computer or smart watch. The computer program may be universally usable with a large variety of portable electronic devices, e.g. provided by a variety of manufacturers and/or including a variety of different or differently configured portable electronic devices. The computer program may be universally usable or executable by a variety of portable electronic devices that distinguish by their hardware and/or software configurations. Hence, the computer program may be executable by a first type or class of portable electronic devices comprising a camera and an electronic reader, which are arranged at a first spatial offset relative to each other. Here, the respective spatial image offset information may be provided by the computer program, e.g. on the basis of a hardware recognition of the portable electronic device.

Likewise, the computer program may be also executable by a second type or class of portable electronic devices, wherein the electronic reader and the camera are arranged in a second spatial offset relative to each other, which distinguishes from the first spatial offset. Also here, and by obtaining spatial image offset information, being indicative of the specific hardware implementation of the portable electronic device, the computer program can be adapted accordingly, so as to correctly display the virtual outline of the medical device on the electronic display of the portable electronic device.

Generally, the scope of the present disclosure is defined by the content of the claims. The portable electronic device, the system, the method and the computer program as described herein are in no way limited to specific embodiments or examples but comprise any combination of elements of different embodiments or examples. Insofar, the present disclosure also covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20 °C), or refrigerated temperatures (e.g., from about −4 °C to about 4 °C). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome. Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

### BRIEF DESCRIPTION OF THE DRAWINGS

In the following, numerous examples of a portable electronic device to co-operate with a medical device and a method of reading a machine-readable identifier of the medical device will be described in greater detail by making reference to the drawings, in which:

FIG. 1 schematically illustrates an example of a portable electronic device,

FIG. 2 shows a user making use of the portable electronic device to read a machine-readable identifier as provided on or inside a medical device,

FIG. 3 shows the electronic display of the portable electronic device in a first stage of mutually orienting or aligning the electronic reader relative to the machine-readable identifier,

FIG. 4 shows the electronic display of FIG. 3 at a second stage of mutual alignment between the machine-readable identifier and the electronic reader, and

FIG. 5 shows a further stage of the alignment procedure, when the electronic reader correctly aligns with the machine-readable identifier,

FIG. 6 shows a further configuration of a portable electronic device with an electronic display in a first stage of alignment with respect to a medical device,

FIG. 7 shows a further stage of alignment between the medical device and the portable electronic device,

FIG. 8 schematically shows a first configuration of a portable electronic device,

FIG. 9 shows another configuration of another portable electronic device,

FIG. 10 schematically illustrates an example of a label attached or attachable to a housing of a medical device,

FIG. 11 shows another example of a label for attachment to a housing of the medical device,

FIG. 12 shows a further example of the portable electronic device during an alignment procedure,

FIG. 13 shows a side view of the portable electronic device and the medical device in a first stage of alignment,

FIG. 14 shows the example of FIG. 13 in a second stage of alignment,

FIG. 15 is a block diagram of portable electronic device,

FIG. 16 is a block diagram of a data structure of data to be stored in a memory of the portable electronic device,

FIG. 17 shows a flowchart of a method of reading a machine-readable identifier and

FIG. 18 illustrates another flowchart of the method of reading the machine-readable identifier.

### DETAILED DESCRIPTION

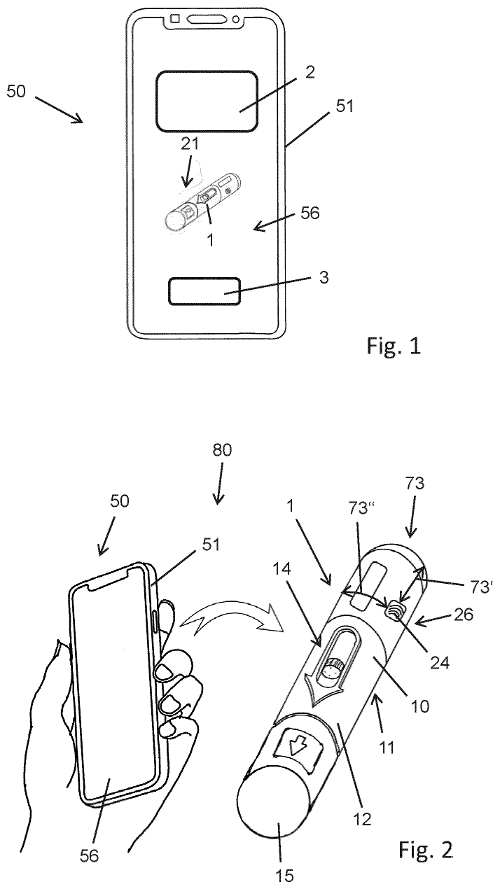

In FIG. 1 there is illustrated one example of a portable electronic device 50. The electronic device 50 comprises a housing 51, a camera 52, typically provided on a lower side or backside, e.g. as illustrated in FIGS. 8 and 9 and an electronic display 56, typically provided on an opposite front side.

The electronic device 50 may be implemented as a portable electronic device. It may be implemented as a smartphone, as a smart watch or as a tablet computer. As particularly illustrated in FIG. 2, the electronic device 50 can be implemented as a hand held mobile electronic device 50. On the electronic display 56 there may be provided an input section 3, where a user may tap a confirmation of where a user may enter respective commands. Typically, the electronic display 56 is implemented as a touch sensitive display. It may comprise a dedicated display section 2, or display window, in which particular information, e.g. about a medical device 1 may be visually displayed.

The camera 52 can be connected to the electronic display 56 in a signal transferring manner, e.g. via a processor 60. The processor 60 may comprise an image or graphics processor, such that electronic signals generated by or obtainable from the camera 52 and processed by the processor 60 cause a generation of a visual camera image on the electronic display 56, e.g. in a camera window 7 on the electronic display 56.

As particularly illustrated in FIG. 1, the electronic display 56 is operable to reproduce an image 21 of the medical device 1, namely when the medical device 1 is captured by the camera 52. Typically, the camera 52, the processor 60 and the electronic display 56 are operable to provide a live image 21 of the medical device 1 on the display 56.

The medical device 1 may comprise an injection device 12. The injection device 12 comprises a housing 10 with an outline 11. The housing 10 may accommodate a medicament container 14, e.g. in form of a cartridge comprising a tubular-shaped barrel and sealed towards an outlet by a closure, such as a septum. The medicament container may be provided with a piston slidably received inside the tubular shaped barrel of the medicament container 14. The injection device 12 may comprise a drive mechanism operable to exert a distally directed pressure onto the piston of the medicament container so as to expel a well-defined amount of the medicament, i.e. a dose of the medicament from the medicament container through an injection needle into a portion of a user's or patient's skin, which is punctured or penetrated by the injection needle (not illustrated).

The injection device 12 and in particular the injection outlet thereof may be equipped with a protective cap 15, which needs to be disassembled to expose the injection needle.

At least one of the medicament container 14 and the housing 10 of the injection device 12 is provided with a machine-readable identifier 24. The machine-readable identifier 24 may comprise a NFC tag 26. The portable electronic device 50 may be equipped with a corresponding electronic reader 54, which is operable to read information stored in the machine-readable identifier 24. Hence, the electronic reader may be implemented as a NFC reader or NFC transceiver. For reading information from the machine-readable identifier 24 it is necessary to align the portable electronic device 50 in a well-defined or predefined position and/or orientation relative to the medical device 1.

Only when the electronic reader 54 of the portable electronic device 50 is in reading distance or in a reading alignment or reading orientation with regards to the machine-readable identifier 24 a reading of information stored in the machine-readable identifier 24 will be possible by the electronic reader 54.

The portable electronic device 50 is equipped or provided with a guiding function or user assisting function and is hence operable to assist or to guide a user to correctly align the portable electronic device 50 relative to the medical device 1 to arrive in a mutual configuration, position or orientation, in which the electronic reader 54 is correctly aligned with the machine-readable identifier 24 and in which configuration the information stored in the machine-readable identifier can be read out.

The medical device 1 and the electronic device 50 belong to a system 80 or constitute a system 80, which is operable to provide an assisted alignment of the machine-readable identifier 24 and the corresponding electronic reader 54.

In some examples, the information stored in the machine-readable identifier comprises at least one of the following information: medicament name, medicament type, medicament amount, production date of the medicament, production site of the medicament, best before date of the medicament, pharmaceutical substance of the medicament, LOT number of the charge of medicament production.

The portable electronic device for 50 may assist or instruct a user in using the medical device 1 or injection device 12. The computer program and hence a software or software app deployed or installed with the electronic device 50 may be operable to log and/or to monitor a one-time or repeated use of the medical device 1 or injections device 12, either automatically or through interaction with a user. Hence, a user may simply indicate, e.g. via touching in the input section 3, that he is about to inject a dose of the medicament by making use of the injection device 12.

By way of an automated readout of data stored in the machine-readable identifier 24, the portable electronic device 50 may precisely gather and collect data, which medicament was or is currently used at which time by the user or patient.

As becomes apparent from the two configurations as shown in FIGS. 8 and 9, the position of the electronic reader 54 may distinguish from the position of the camera 52 with regards to the geometry of the housing 51 of the electronic device 50. As shown in FIG. 8, the electronic reader 54 may be located at first and second transverse offsets 71, 72 from the camera 52. There may be provided a two-dimensional spatial imaging offset 71, 72 between the position of the camera 52 and the position of the electronic reader 54 with respect to the housing 51 of the electronic device 50.

With the configuration of the electronic device 50′ FIG. 9, there may be different positions for both of the camera 52 and the electronic reader 54 compared with the configuration of the electronic device 50 according to FIG. 8. The electronic device 50′ as illustrated in FIG. 9 may be a different electronic device compared to the electronic device 50 as shown in FIG. 8. The electronic devices 50, 50′ as shown in FIGS. 8 and 9 may be provided by different manufacturers or may represent portable electronic devices 50 of different type or configuration.

Accordingly, the electronic device 50 as shown in FIG. 8 may be provided with spatial image offset information 71 one being indicative of a spatial distance or spatial offset between the electronic reader 54 and the camera 52 relative to each other answers or relative to the housing 51. Specifically, the camera 52 may be offset from the reader by a distance 71′ along a first direction and may be offset from the reader by a distance 71″ along a second direction, wherein the first and second directions are perpendicular to each other.

With the electronic device 50′ as shown in FIG. 9, the spatial image offset information 72 reflects a different configuration of a spatial offset or spatial arrangement between the electronic reader 54 and the camera 52, typically provided at or on a planar-shaped backside of the housing 51 opposite the electronic display 56.

In order to establish or to enable reading of information from the machine-readable identifier 24 as provided in or on the housing 10 of the medical device 1 or injection devices 12 it may be necessary to bring the electronic reader 54 and the machine-readable identifier 24 in mutual alignment and/or in close vicinity to each other.

Since the position of the electronic reader 54 may not be directly visible to a user of the electronic device 50 and since the position of the electronic reader 54 may change depending on the specific model or type of the portable electronic device 50 it may be rather difficult to obtain a correct alignment enabling or allowing to read the information stored in the machine-readable identifier 24.

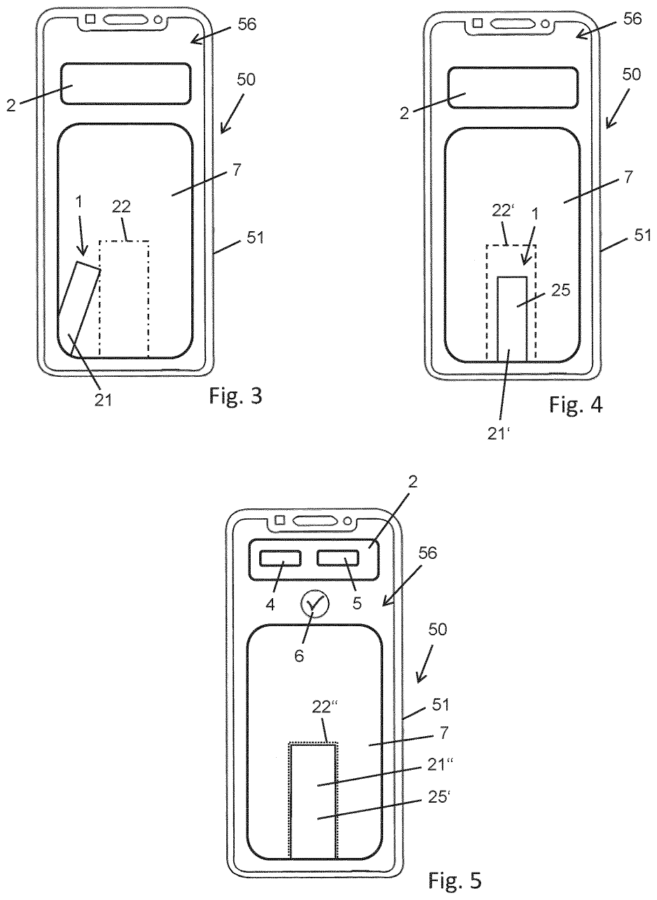

The portable electronic device 50, the camera 52, the electronic display 56 and the processor 60 are particularly operable to provide an alignment and a user guiding functionality, which by way of an example, is schematically illustrated in the sequence of FIGS. 3-5.

The portable electronic device 50 and associated computer program of software application executed by the processor 60 of the portable electronic device 50 is or are configured to be used with a particular type of a medical device 1, which is known to the user. In other words, the portable electronic device is set up for conducting a data readout of a machine-readable identifier 24 provided on a particular and pre-defined type of medical device 1 or injection device 12. Hence, the software application executed by the processor 60 is deployed, calibrated or tuned to a specific type of a medical device 1.

The processor 60 is connected to the camera 52 and to the electronic display 56. It is operable to visualize a captured image 21 of the medical device 1 when the medical device 1 is captured by the camera 52 of the portable electronic device 50.

Concurrently, the processor 60 is operable to display a virtual outline and hence a target position or target orientation of the medical device 1 on the electronic display. The virtual outline 22 may be separately provided on the electronic display 56 and may define or visualize a predefined target position and/or a predefined target orientation for the captured image 21 of the medical device 1.

In the configuration as illustrated in FIG. 3 the camera 52 captures a portion of the injection device 12 or medical device 1, which is not yet correctly aligned and which is hence oriented at a certain angle relative to the elongation of the virtual outline 22 of the medical device 1.

In the further illustration of FIG. 4, the user has turned and/or reoriented the medical device 1 to such a degree, such that the captured image 21′ of at least a portion of the medical device 1 is substantially co-aligned with the virtual outline 22′ as provided concurrently on the electronic display 56. As it is immediately apparent from FIG. 4, the structure and/or the geometry and hence the outer circumference of the captured image 21′ is still somewhat smaller compared to the virtual outline 22′ of the medical device 1. This is an indication, that the distance between the housing 51 and the medical device 1 is still too large.

Bringing the medical device 1 or the injection device 12 closer to the housing 51 of the portable electronic device 50 leads to a respective increase of the captured image 21″ on the electronic display 56 until the captured image 21″ matches with virtual outline 22″. When reaching a substantial overlap between the captured image 21″ and the virtual outline 22″ the machine-readable identifier 24 is correctly aligned with the electronic reader 54. In this configuration as e.g. shown in FIG. 5 a readout of the data or information stored in the machine-readable identifier 24 is enabled and the electronic reader 54 can actually readout the respective information.

In some examples, the virtual outline 22 is visually illustrated on the electronic display 56 concurrently with a live image of the camera 52. This way, the user is provided with a direct and immediate feedback on how to arrange the medical device 1 relative to the portable electronic device 50 to obtain the substantially overlapping configuration as e.g. shown in FIG. 5.

In some examples, the processor 60 may be operable to recognize at least one of a position and an orientation of the medical device 1 in the captured image 21. For instance, and as illustrated in FIG. 4, the processor 60 may be operable to recognize a so-called recognized item 25 in the image 21′ as illustrated on the electronic display 56 of FIG. 4. Also, the processor 60 may be operable to evaluate that the recognized item 25 does not yet match with the virtual outline 22′. Insofar, the processor may not only recognize a pre-defined item 25 in the image 21 but may be further operable to determine a degree of overlapping between the recognized item 25 and the virtual outline 22.

Concurrent with the recognition of the medical device 1 as a recognized item 25 in the captured image 21, the processor 60 may be operable to modify the visual appearance of the virtual outline 22. In a situation as e.g. shown in FIG. 3, where the captured image 21 has not yet been recognized as a medical device 1 or as an injection device 12, the virtual outline 22 is represented in a dotted structure. Upon recognition the recognized item 25 the virtual outline 22 may switch into the virtual outline 22′ as shown in FIG. 4. Here and in response to the recognition of the recognized item 25 the processor 60 may be operable to modify the visual appearance of the virtual outline 22 from the dash dotted illustration as shown in FIG. 3 towards a dashed structure of the virtual outline 22 as shown in FIG. 4.

Furthermore and when reaching an effective overlapping configuration as e.g. shown in FIG. 5 the processor 60 may be further operable to change the visual appearance of the virtual outline 22′ towards the virtual outline 22″ as shown in FIG. 5. Here, the structure of the virtual outline 22′ as shown in FIG. 4 changes to the virtual outline 22″ reproduced as a dotted structure.

In the present illustration of FIGS. 3-5, the different appearances of the virtual outline 20, 22′, 22″ may help and guide a user to conduct and to achieve a predefined mutual alignment of the electronic reader 54 with respect to the machine-readable identifier 24.

The different illustrations of the virtual outline 20, 22′, 22″ may be reflected by different colors of the virtual outline, by a variation of the brightness or contrast as well as with regards to a temporal appearance of the virtual outline 22, 22′, 22″. Hence, the different illustrations of the virtual outlines 20, 22′, 22″ as shown in FIGS. 3-5 may be also reflected by a constant or intermittent appearance or by a kind of a blinking of the virtual outlines 22, 22′, 22″.

Also here, a variation of the frequency of the appearance and disappearance of the virtual outlines 22, 22′, 22″ may indicate to a user that the degree of alignment and/or the degree of overlapping between the recognized item 25 and the virtual outline 22 increases or decreases.

Once a correct or predefined alignment has been achieved, as e.g. illustrated in FIG. 5, the electronic reader 54 is operable to read out the data or information stored in the machine-readable identifier 24. The respective information, such as medicament type, medicament amount, prescription information, manufacturing date, manufacturing site, LOT number, best before date may be visualized in information windows 4, 5 of a display section 2 as provided on the electronic display 56. There may be further provided a confirmation portion 6, which may be emulated by the processor 60 on the touch sensitive electronic display 56. Here, a user may simply touch the confirmation 6 as to prompt or to confirm that the data as stored in the machine-readable identification 24 has been successfully captured by the electronic device 50.

The illustration of the virtual outline 22 on the electronic display 56 may strongly depend on the software or hardware configuration of the portable electronic device 50. It may strongly depend on the relative position or orientation of the electronic reader 54 and the camera 52 as e.g. illustrated with the two examples of FIGS. 8 and 9. For instance, the scenario as illustrated in the sequence of FIGS. 3-5 may be obtained with a portable electronic device 50 as shown in FIG. 8 with a first spatial image offset 71 between the camera 52 and the machine-readable identifier 54.

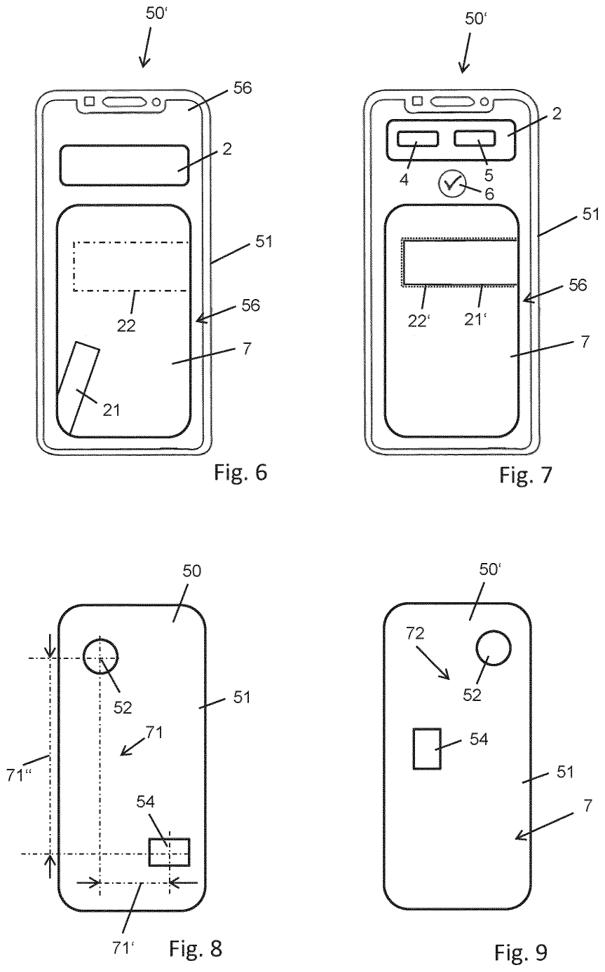

With another configuration of a portable electronic device 50′ as shown in FIG. 9 there may be provided a virtual outline 22 as shown in FIG. 6. Here and compared to the configurations as shown in FIGS. 3-5, the desired or intended orientation and/or position of the captured image 21 significantly changes compared to the configuration as shown in FIGS. 3-5.

Here, the longitudinal axis of the e.g. tubular shaped injection device 12 or medical device 1 has to be rotated by about 90° relative to the housing 51 of the electronic device 50′ compared to the configuration as shown in FIGS. 3-5. Also here, and due to the kind of augmented reality provided in the display window 7 of the electronic display 56 the user will have only little difficulties in correctly aligning the captured image 21 with the virtual outline 22 as provided in the camera window 7.

In the configuration according to FIG. 6 it is immediately apparent to the user, that the medical device 1 has to be rotated counterclockwise by about 90°-115° and that the distance between the housing 51 and the medical device 1 has to be reduced. At the end and when there is provided a substantial spatial and virtual overlap between the captured image 21′ with the virtual outline 22′ as shown in FIG. 7, a respective wireless read-out of the machine-readable identifier 24 can be provided.

Similarly, and as already explained in connection with FIG. 5 there may show up respective information 4, 5 in a display section 2 or information window of the electronic display 56.

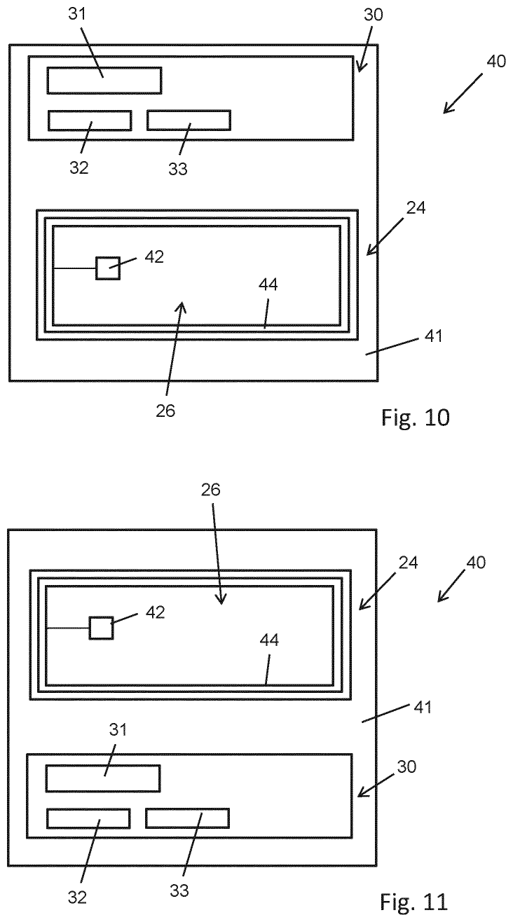

In FIGS. 10 and 11 there are shown two examples of a label 40, which may be provided or adhered to at least one of the medicament container 14 and the housing 10 of the medical device 1. The label 40 may comprise a planar and pliable or flexible substrate 41, such as a bendable or pliable foil. The substrate 41 may comprise an adhesive foil, which may be fixedly attached to one of the housing 10 and the medicament container 14 accommodated inside the housing 10.

The label 40 may be provided with a visual mark 30, comprising or providing numerous visual information 31, 32, 33. The information 31, 32, 33 may be provided in printed or visual form on the surface of the visual mark 30. The visual mark 30 may be a printed label adhered to or attached to the substrate 41.

The label 40 is further provided with the machine-readable identifier 24, which may comprise a NFC tag 26 or RFID tag. It is typically provided with a tag processor 42 and an antenna 44, electrically connected to the tag processor 42. The machine-readable identifier 24, e.g. the NFC tag 26 may be implemented as a passive transceiver, which is operable to withdraw electric energy from an RF field of an electronic reader 54 of an electronic device 50 thus allowing to power up the tag processor 42 and to exchange or to transmit data to the electronic reader 54.

In the example of the label 40 according to FIG. 10 the machine-readable identifier 24 is located below and/or next to the visual mark 30. In the further configuration of a label 40 according to FIG. 11 there is provided another configuration or mutual arrangement of the visual mark 30 and the machine-readable identifier 24. There, the machine-readable identifier 24 is arranged above and/or next to the visual mark 30. In effect, the positions of the visual mark 30 and the machine-readable identifier machine-readable identifier 24 in the example of FIG. 10 have interchanged compared with the respective positions of the visual mark 30 and the machine-readable identifier 24 of the example as shown in FIG. 11.

The portable electronic device 50 and/or the computer program to be executed by the processor 60 may be provided with respective information, which kind of label 40 is expected to be provided on or inside the housing 10 of the medical device 1 or injection device 12. With this information it is even conceivable that the processor 60 does not only provide a virtual outline 22 of the medical device 1 on the electronic display 56. Rather, the processor 60 may be also or alternatively operable to visualize the captured image 21 of the medical device 1 concurrently with a transparent or semi-transparent virtual image 23 of the medical device 1 on the electronic display 56.

With the transparent or semi-transparent virtual image 23 provided concurrently with the captured image 21 of the medical device 1 also a visual mark 30 as provided on the housing 12 may be used to correctly align and/or to correctly orient the medical device 1 relative to the electronic reader 54.

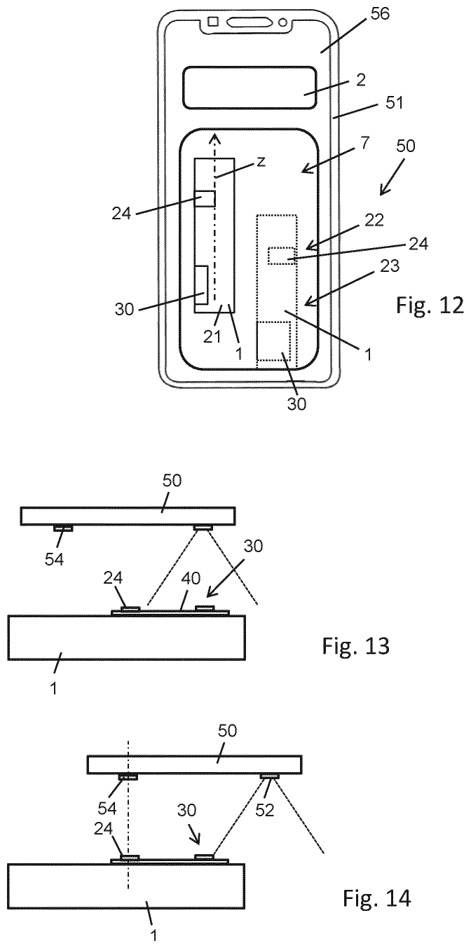

Further, and as shown in FIG. 12, the captured image 21 of the medical device 1 only shows the visual mark 30 and/or the identifier 24 on the captured image 21. The transparent or semi-transparent virtual image 23 of the medical device 1 indicates to the user, that the medical device 1 has to be rotated with regards to its longitudinal axis (z) as an axis of rotation for not only bringing the captured image 21 in a substantial overlapping configuration with the virtual outline 22 but also to rotate the medical device 1 by a predefined angle so that the visual mark 30 substantially aligns or overlaps with the visual mark 30 of the transparent or semi-transparent virtual image 23 as reproduced or provided on the electronic display 56. This way there can be even enabled and provided a three-dimensional alignment between the electronic reader 54 and the machine-readable identifier 24.

In FIGS. 13 and 14, the process of correctly aligning the portable electronic device 50 relative to the medical device 1 is shown on a side view. In the configuration of FIG. 13 there is a particular alignment mismatch between the machine-readable identifier 24 and the electronic reader 54 of the electronic device 50 at least with regard to the horizontal direction. The camera 52 is operable to visualize at least the visual mark 30, which may then appear, e.g. in the middle of the electronic display 56.

In the configuration of FIG. 13, the processor 60 will be operable to show a respective alignment mismatch on the electronic display 56. Here, the captured image 21 of the medical device 1 may be visualized at a certain offset from the virtual outline 22 of the medical device 1. This offset visualization as provided by the electronic display 56 may instruct the user to move the electronic device 50 in a horizontal direction relative to the medical device 1 until a respective matching configuration is obtained, which reflects the intended alignment of the machine-readable identifier 24 of the medical device 1 relative to the electronic reader 54 of the portable electronic device 50 as shown in FIG. 14.

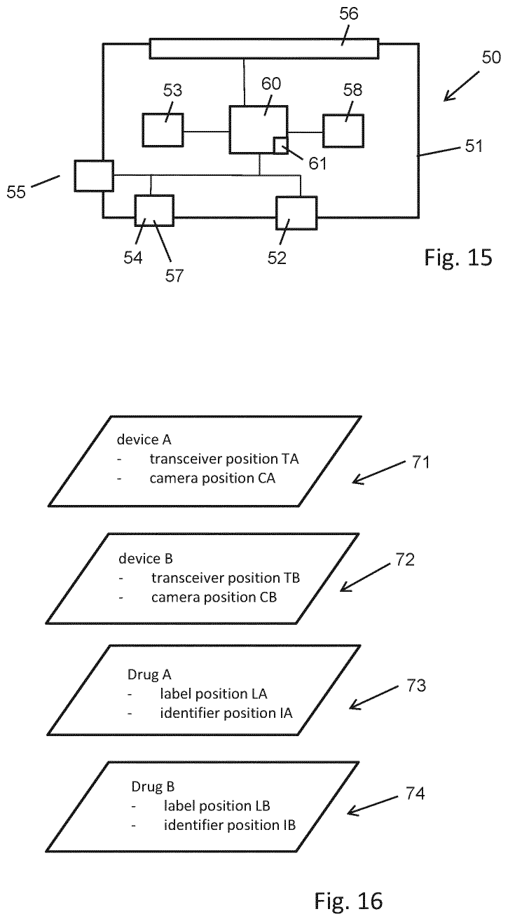

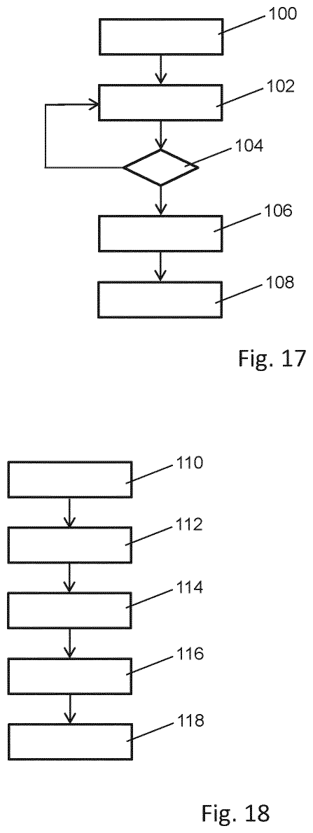

In FIG. 15 and by way of example numerous hardware components of the portable electronic device 50 are schematically illustrated. The portable electronic device 50 comprises a housing 51 and a display 56, e.g. provided on one side of the housing 51. On an opposite side of the housing, e.g. on a bottom or underside of the housing 51 there is typically provided the camera 52. Optionally, the electronic reader 54 may be also provided on or near the underside. The electronic reader 54 may be implemented as a NFC transceiver 57.

Optionally, the electronic device 50 comprises a short range transceiver 55, e.g. in form of a Bluetooth or Wi-Fi transceiver by way of which the electronic device 50 may set up another communication link with further communication hardware or communication infrastructure.

The electronic device 50 further comprises a battery 53 to provide electrical power for the processor 60 and for the transceivers 55, 57. The processor 60 may be provided with an electronic device identifier 61. The electronic device identifier 61 is particularly operable to store and/or to provide spatial image offset information 71, 72, which is indicative of a spatial offset between the camera 52 and the electronic reader 54. Insofar as these methods are implemented in a computer program that can be installed on different types of electronic devices (e.g., different types of smartphones), the spatial image offset information 71, 72, may be selected from a database (stored, e.g., on the electronic device or elsewhere) and/or could be embodied in the computer program that includes spatial offset information for a variety of electronic devices. This way, an app or computer program suitable for a variety of different electronic devices, e.g. different smartphones, could contain the spatial offset information for each type of electronic device. Depending on the type of electronic device on which the app or computer program is installed or deployed, the spatial offset information pertinent to the specific electronic device or smart phone can be selected and used for suitably calibrating the electronic device.

Alternatively, the electronic device identifier 61 may be provided in a non-volatile portion of the memory 58. The memory 58 may be further provided with memory blocks either of volatile or non-volatile type. The memory 58 is operable to store at least information or data as obtained by the readout of the machine-readable identifier 24 as provided by the electronic reader 54.

In FIG. 16 there are visibly illustrated numerous data or data structures that can be stored in the memory 58. Here, spatial image offset information 71 one may be indicative of a transceiver position TA and a camera position CA, either relative to each other or in relation to a housing 51 of a first electronic device A 50.

With another electronic device 50′, e.g. provided by another manufacturer or with an electronic device 50′ of a different type the spatial image offset in formation 72 may be indicative of a transceiver position TB and a camera position CB, either relative to each other or in relation to the housing 51 of the further electronic device B 50′.

In the same or like manner the memory 58 may be provided with spatial device offset information 73, 74, which device offset information 73, 74 is indicative of a spatial offset between the machine-readable identifier 24 and at least one of a visual mark 30 on the medical device 1 and an outline 11 of the medical device 1. Here, a first spatial device offset information 73 may be indicative of a label position LA and/or of an identifier position IA of a first medical device A 1 or of a first injection device A 12.

The spatial device offset information 74 as indicated in FIG. 16 may be indicative of another label position LB and another identifier position IB of another drug B as provided in another medicament container 14 or in another medical device 1′ or injection device 12′.

A further example of spatial device offset information 73 is shown in FIG. 2. There, the machine-readable identifier 24 is implemented as a NFC tag 26 and is provided on an outside surface of the housing 10 of the injection device 12. It is located at a predefined longitudinal distance 73′ from a proximal end of the housing 10 and is arranged at a predefined tangential distance 73″ from a side edge or characteristic portion (e.g., a marking or physical feature like a ridge) of the housing 10.

When initializing the portable electronic device 50 and/or when using a respective computer program deployed by the portable electronic device 50 for the first time, the computer program may be operable to obtain or to read the electronic device identifier 61, thereby obtaining respective spatial offset information 71, 72 being indicative of a spatial offset between the camera 52 and the electronic reader 54.

This way, the portable electronic device 50 can be correctly calibrated or adjusted to be used with a particular type of a medical device 1. In the same or like manner and when for instance not only the virtual outline 11 but when also a visual mark 30 as provided on an outside of the medical device 1 should be used for the alignment between the electronic reader 54 and the machine-readable 24 it is of particular benefit to provide or to select the spatial device offset information 73, 74, which is typically stored or provided by the computer program for all types of available medical devices 1 to be used with the portable electronic device 50.

In FIG. 18, there is illustrated a flowchart of a method of reading a machine-readable identifier 24 as provided in or on a medical device 1 as described herein. Here and in a first step 100 a user may determine or select a particular type of medical device 1 to be used with the portable electronic device 50. In a subsequent step 102, the camera 52 is activated and a camera image is reproduced in a camera window 7 of the electronic display 56. Concurrently, the virtual outline 22 is illustrated by the processor 60 in the camera window 7.

In step 104, it is checked if and in how far the captured image 21 of the medical device 1 as obtained by the camera 52 overlaps or aligns with the virtual outline 22. As long as there is an insufficient degree of overlapping the method returns to step 102 and repeatedly conducts the steps 102 and 104 until the captured image 21 of the medical device 1 suitably overlaps with the virtual outline 22 on the electronic display 56. In a subsequent step 106 and after a suitable degree of overlapping has been reached there may be optionally provided a user feedback, e.g. by changing the appearance of the virtual outline 22, thereby indicating to a user, that a sufficient degree of overlapping between the captured image 21 and the virtual outline 22 has been obtained. In a subsequent step 108 the machine-readable identifier 24, e.g. the electronic label is read out by the electronic reader 54. Concurrently or thereafter, there is illustrated respective information on the electronic display 56.

In the further flowchart as illustrated in FIG. 18, the numerous steps of setting up or deploying the portable electronic device 50 for reading the machine-readable identifier 24 and for assisting a user in correctly aligning the portable electronic device 50 and the medical device 1 is schematically illustrated. Here, and in a first step 110, the electronic device identifier 61 being indicative of a hardware configuration and being particularly indicative of a spatial offset information 71, 72 being indicated of a spatial offset between the camera 52 and the electronic reader 54 of the respective portable electronic device 50 is read out, e.g. by the processor 60.

Thereafter and in step 112 there is conducted a kind of a calibration of guiding and assistance function on the basis of the offset information between the camera 52 and the electronic reader for 54. Optionally, and in step 114 there is derived a relation between a spatial offset between the machine-readable identifier 24 and at least one of the visual mark 30 and an outline 11 of the medical device 1.

Depending on the derivation of respective spatial relations between the camera 52, the electronic reader 54 and the visual mark 30, the machine-readable identifier 24 is aligned relative to the electronic reader 54 in step 116 in a way as described above in connection with steps 112, 114. Here, the user moves the portable electronic device 50 relative to the medical device 1 as long as required and guided by the augmented visualization on the electronic display 56 until the electronic reader 54 is correctly aligned with the machine-readable identifier 24 in step 118.

### REFERENCE NUMBERS

1 medical device
2 display section
3 input section
4 information
5 information
6 confirmation
7 camera window
10 housing
11 outline
12 injection device
14 medicament container
15 cap
21 image
22 virtual outline
23 virtual image
24 identifier
25 recognized item
26 NFC tag
30 visual mark
31 information
32 information
33 information
40 label
41 substrate
42 tag processor
44 antenna
50 electronic device
51 housing
52 camera
53 battery
54 electronic reader
55 local range transceiver
56 display
57 NFC transceiver
58 memory
60 processor
61 electronic device identifier
80 system

1-15. (canceled)

16. A portable electronic device comprising:
a camera operable to capture an image of a medical device, wherein the medical device is provided with a machine-readable identifier,
an electronic reader operable to read the machine-readable identifier when aligned with the machine-readable identifier,
an electronic display operable to visualize the captured image of the medical device and
a processor connected to the camera and connected to the electronic display, the processor being operable to display a virtual outline of the medical device on the electronic display, such that, when the electronic reader is aligned with the machine-readable identifier, the virtual outline aligns and/or overlaps with the captured image on the electronic display.

17. The portable electronic device according to claim 16, further comprising an electronic device identifier containing or providing spatial image offset information being indicative of a spatial offset between the camera and the electronic reader.

18. The portable electronic device according to claim 16, further comprising a memory operable to store spatial device offset information being indicative of a spatial offset between the machine-readable identifier and at least one of a visual mark on the medical device and an outline of the medical device.

19. The portable electronic device according to claim 17, wherein the processor is operable to display the virtual outline of the medical device on the electronic display with respect to at least one of the spatial image offset information and the spatial device offset information.

20. The portable electronic device according to claim 16, wherein the processor is operable to recognize at least one of a position and an orientation of at least one of the medical device and the electronic identifier in the captured image as a recognized item.

21. The portable electronic device according to claim 20, wherein the processor is operable to modify an appearance of the virtual outline on the electronic display depending on a degree of alignment and/or a degree of overlapping between the recognized item and the virtual outline.

22. The portable electronic device according to claim 21, wherein the processor is operable to modify at least one of a structure, a color, a brightness, a contrast and a temporal appearance of the virtual outline in response to a variation of the degree of alignment and/or overlapping between the recognized item and the virtual outline.

23. The portable electronic device according to claim 16, wherein processor is further operable to display a transparent or semi-transparent virtual image of the medical device on the electronic display, such that, when the electronic reader is aligned with the machine-readable identifier, the virtual image aligns and/or overlaps with the captured image on the electronic display.

24. The portable electronic device according to claim 16, wherein the electronic reader comprises a near field communication NFC transceiver.

25. A system comprising:
a medical device, the medical device comprising a housing and a machine-readable identifier on or inside the housing,
a portable electronic device comprising
a camera operable to capture an image of a medical device, wherein the medical device is provided with a machine-readable identifier,
an electronic reader operable to read the machine-readable identifier of the medical device when aligned with the machine-readable identifier,
an electronic display operable to visualize the captured image of the medical device and
a processor connected to the camera and connected to the electronic display, the processor being operable to display a virtual outline of the medical device on the electronic display, such that, when the electronic reader is aligned with the machine-readable identifier, the virtual outline aligns and/or overlaps with the captured image on the electronic display.

26. The system according to claim 25, wherein the medical device comprises an injection device.

27. A method of reading a machine-readable identifier provided in or on a medical device with a portable electronic device, wherein the portable electronic device comprises a camera, an electronic reader and an electronic display, the method comprising the steps of:
capturing an image of the medical device with the camera of the portable electronic device,
displaying a virtual outline of the medical device on the electronic display,
visualizing the captured image of the medical device on the electronic display,
moving the medical device relative to the portable electronic device to align and/or to at least partially overlap the captured image with the virtual outline of the medical device and thereby bringing the electronic reader into one of a reading distance or reading orientation with respect to the machine-readable identifier.

28. The method according to claim 27, further comprising the steps of:
obtaining or acquiring of at least one of spatial image offset information and a spatial device offset information, wherein the spatial image offset information being indicative of a spatial offset between the camera and the electronic reader and wherein the spatial device offset information being indicative of a spatial offset between the machine-readable identifier and at least one of a visual mark on the medical device and an outline of the medical device and
displaying the virtual outline of the medical device on the electronic display with respect to at least one of the spatial image offset information and the spatial device offset information.

29. The method according to claim 28, further comprising the step of:
recognizing at least one of a position and an orientation of at least one of the medical device and the electronic identifier in the captured image as a recognized item.

30. A computer program comprising computer executable instructions, which when executed by a processor of a portable electronic device comprising
a camera operable to capture an image of a medical device, wherein the medical device is provided with a machine-readable identifier,
an electronic reader operable to read the machine-readable identifier when aligned with the machine-readable identifier,
an electronic display operable to visualize the captured image of the medical device and
a processor connected to the camera and connected to the electronic display, the processor being operable to display a virtual outline of the medical device on the electronic display, such that, when the electronic reader is aligned with the machine-readable identifier, the virtual outline aligns and/or overlaps with the captured image on the electronic display, causes the processor to:
capture an image of the medical device with the camera of the portable electronic device
display a virtual outline of the medical device on the electronic display, and
visualize the captured image of the medical device on the electronic display concurrent with the virtual outline of the medical device.
